(19) United States
(12) Patent Application Publication
(10) Pub. No.: US 2019/0374301 A1
(43) Pub. Date: Dec. 12, 2019

(54) SIMPLIFIED STERILE BAG WITH PACKAGING AND METHOD FOR REDUCING DEPLOYMENT STEPS

(71) Applicant: Canon U.S.A., Inc., Melville, NY (US)

(72) Inventor: Alexander Altshuler, Cambridge, MA (US)

(21) Appl. No.: 16/004,213

(22) Filed: Jun. 8, 2018

(51) Int. Cl. A61B 46/10; A61B 50/30

(52) U.S. Cl. CPC A61B 46/10; A61B 50/30

(57) ABSTRACT

A simplified sterile contamination prevention bag assembly and method for reducing deployment steps is provided for covering a medical instrument deployable from a system having a deployment port for deploying the instrument from a non-deployed position to a deployed position through the deployment port and externally distal from the deployment port. The bag assembly includes a bag including a deployment port attaching end configured to be attached to a mating portion of the deployment port and a distal deployment end having a distal mating feature configured to receive a distal portion of the medical instrument. The bag is folded in a non-deployed state, and unfolded when the medical instrument is moved from the non-deployed position to the deployed position.

### FIELD OF THE DISCLOSURE

The subject disclosure relates to sterile bags and/or drapes for equipment used in a medical and/or biological setting. Moreover, the subject disclosure relates to packaging and deployment of sterile bags and drapes in a sterile environment by one person and/or multiple person deployment.

### BACKGROUND INFORMATION

A large number of current medical and biological procedures require a sterile bag or drape to prevent cross contamination between a patient/sample and a device used for the procedure. For elongated devices the bag/drape used is usually a polymer film tube connected to the proximal end of non-sterile portion of the device and extending to at least the end of required sterile field enclosing the device. As a new bag/drape must be deployed for each such procedure the ease and expedience of its deployment is often essential for the efficient work flow.

A first prior art example of a sterile bag/drape for the medical field is taught in U.S. Patent Application Publication No. U.S. No. 2014/0338676 A1, entitled “Medical Drape And Methods Of Covering Equipment With Medical Drapes” to Marinchak. A sterile drape 4 is provided to cover medical equipment 8, 10, 36. The drape 4 may include an expandable element 30 to open the open end of the drape 16. A sheath may also be provided to cover the drape 4 prior to use. The drape 4 may also be held in a container, which may be coupled to the equipment. Furthermore. the drape 4 may include one or more partially deployed positions in which the drape 4 is partially deployed yet the sterile surface of the drape 4 remains unexposed so that the drape 4 may be partially deployed by non-sterile personnel and stored in a non-sterile area.

A second prior art example of sterile bag/drape for the medical field is taught in U.S. Pat. No. 5,792,045, entitled “Sterile Surgical Coupler And Drape” to Adair. An apparatus and method are provided for enclosing a non-sterile camera setup comprising a video camera VC, its trailing cables, and a standard optical connector OC for use of the unsterile camera setup in the sterile environment of an operating room. The apparatus includes a coupler having a first end for attachment to the unsterile camera setup VC. FR. OC and a second end for attachment to a sterile endoscope E. A passageway is formed inside the coupler 10 that extends from the first end to the second end thereof providing an optical pathway whereby an image from the endoscope E may be transmitted to the unsterile camera setup VC, FR. OC. A transparent window is mounted transversely across the passageway between the first and second ends of the coupler providing a sterile barrier therebetween. In operation, a sterile drape 60 is positioned over the first end of the coupler and extends over the unsterile camera setup VC. FR. OC. The sterile drape 60 is secured between the first and second ends of the coupler by a locking ring which secures the distal end of the drape so that a fluid and airtight seal is formed therebetween. A sterile disposable wand may be releasably attached to the second end of the coupler for providing support to the unsterile camera setup VC, FR. OC as it is attached to the coupler 10. The wand may then be detached enabling connection of the sterile endoscope E to the second end of the coupler 10.

A third prior art example of a sterile bag/drape for the medical field is taught in U.S. Pat. No. 8,459,266, entitled “Pleated Bag For Interventional Pullback Systems” to Glynn et al. A system is provided that includes a pullback device, catheter, and pleated bag. Pleated bags 115 are used with interventional pullback systems 5, including imaging catheters such as IVUS catheters, as well as for use with other catheters. An exemplary pleated bag 115 of the system may be an elongate, sterile bag, having a plurality of pleats near the distal end, and including an orifice proximal to the pleats designed to allow passage of a catheter into the bag. The above pleated bag system allows for improved imaging catheter procedures at reduced costs.

A fourth prior art example of a sterile bag/drape used in the medical field is taught in a video uploaded on YouTube.com entitled “P3 medical-Ring-style Camera Drape with elastomeric end section” posted by p3medical (n.d.), and retrieved from link https://www.youtube.com/watch?v=HBENsdvuvpl, herein referred as “P3MEDICAL”. In PSMEDICAL a tube style medical drape is installed on an endoscopic probe. The elastomeric drape is assembled so that it is attached to a ring. The probe is inserted through the ring and then a distally deployed portion of the drape is attached to a proximate end having an attachment coupling. The attachment coupling of the endoscopic probe is then connected to another upstream portion of the medical system. The drape is then unrolled or deployed over the longitudinal length of the upstream portion of the medical system.

One disadvantage to the above prior art is that the bags/drapes are designed for large equipment and methods for covering large medical equipment, as opposed to hand-held devices. See paragraph [0001] of U.S. Patent Application Publication No. U.S. 2014/0338676 A1, entitled “Medical Drape And Methods Of Covering Equipment With Medical Drapes” to Marinchak, for example.

Moreover, as can be observed in the PSMEDICAL video for example, current sterile bag/drape deployment methods are in many cases cumbersome and require coordinated efforts of a sterile and non-sterile person to implement. Hence, typically, multiple personal may be required to deploy complicated drape deployment systems.

And further, despite some advances, such as pre-mounting the sterile bag/drape on the equipment for ease of deployment, sterile drape deployment methods are still in many cases cumbersome, requiring coordinated efforts between at least one sterile and one non-sterile person to implement.

Therefore, a need still exists for improvement and streamlining a process of sterile bag/drape deployment which is simple to use and cost-effective. Ideally, a simple and cost-effective drape system could be deployed by only one person if necessary. Accordingly, in view of these considerations, it would be advantageous to address and/or overcome at least some of the deficiencies described herein above.

### SUMMARY OF EXAMPLE EMBODIMENTS

Accordingly, it can be beneficial to address and/or overcome at least some of the deficiencies indicated herein above, and thus, to provide a simplified sterile drape contamination prevention drape assembly and method for simplified deployment of the same.

According to various example embodiments of the present disclosure, a simplified sterile bag with packaging and method for a simplified one person deployment of the same is provided for covering an medical instrument or device deployable from a deployment port of a system for deploying the instrument.

According to an embodiment of the present disclosure, a contamination prevention bag assembly is provided for covering an instrument deployable from a system having a deployment port for deploying the instrument, the instrument being deployable from a non-deployed position to a deployed position through the deployment port externally distal from the deployment port. The bag assembly includes a bag having an inner and outer bag surface, a deployment port attaching end configured to be attached to a mating portion of the deployment port and a distal deployment end having a distal mating feature configured to receive a distal portion of the instrument. The bag is folded in a non-deployed state. The bag is unfolded when the instrument is moved from the non-deployed position to the deployed position.

According to another embodiment of the present disclosure, an attachment means may be disposed on the deployment port attaching end of the bag so that the deployment port attaching end is configured to be removably attached to the mating portion of the deployment port.

According to yet another embodiment of the present disclosure, the attachment means includes adhesive pre-applied to the deployment port attaching end for attaching the bag to the mating portion of the deployment port.

According to still another embodiment of the present disclosure, the attachment means includes a magnetic flange affixed to the deployment port attaching end that magnetically attaches to the mating portion of the deployment port.

And, according to another embodiment of the present disclosure, the attachment means includes a mechanical means for removably attaching the deployment port attaching end of the bag to the mating portion of the deployment port.

According to still another embodiment of the present disclosure, the contamination prevention bag assembly further comprises contamination prevention packaging hermetically sealing at least the outer bag surface in a non-deployed state, wherein the bag is packaged and stored in a folded state.

According to still yet another embodiment of the present disclosure, the contamination prevention packaging includes at least one removable attachment portion covering the deployment port attaching end of the non-deployed drape assembly, and wherein the at least removable portion is configured to be removed before attaching the attachment means to the mating portion of the deployment port.

Additionally, according to yet another embodiment of the present disclosure, the contamination prevention packaging includes at least one removable inner liner covering the deployment port attaching end of the non-deployed drape assembly, and wherein the at least one removable inner liner portion is configured to be removed before attaching the distal portion of the instrument to the distal mating feature configured to receive the distal portion of the instrument.

And, according to still another embodiment of the present disclosure, the contamination prevention packaging includes at least one removable portion covering at least a distal side and a length side of the bag assembly defined by folded edges formed by the folded bag in the non-deployed state, which when removed, uncovers the distal deployment end of the bag assembly and the folded edges of the bag.

Moreover, according to another embodiment of the present disclosure, a method for utilizing a contamination prevention bag assembly for covering an instrument deployable from a system having a deployment port. The bag assembly includes a bag folded in a non-deployed state, the bag including inner an inner and outer surface, a deployment port attaching end, a distal deployment end having a mating feature configured to receive a distal portion of the instrument, and an attachment means disposed on the deployment port attaching end of the bag; and contamination prevention packaging hermetically sealing at least the outer bag surface in a non-deployed state. Here, the method includes attaching the attachment means of the bag assembly to a mating portion of the deployment port; inserting the distal portion of the instrument through the deployment port attaching end of the bag assembly and engaging the distal portion of the instrument into the distal mating feature configured to receive a distal portion of the instrument; removing the contamination prevention packaging hermetically sealing at least the outer bag surface; and deploying the instrument from a non-deployed position to a deployed position through the deployment port and externally distal from the deployment port, wherein the bag is unfolded when the instrument is moved from the non-deployed position to the deployed position externally distal from the deployment port.

As can be understood from the above description the present disclosure allows for simplified sterile bag/drape deployment coincident and combined with the device deployment saving significant portion of operator (and especially sterile operator) time. Accordingly, a simplified sterile bag/drape with packaging and method for simplified deployment of the same by a single operator and/or more than one operator is realized from the present disclosure.

### BRIEF DESCRIPTION OF THE DRAWINGS

Further aspects, features and advantages of the present disclosure will become apparent from the following detailed description when taken in conjunction with the accompanying figures showing the illustrative numerous embodiments of the present disclosure in which:

FIG. 1 is a cross-sectional view of a simplified sterile bag/drape assembly that has been fully deployed, according to a first example embodiment of the disclosure.

FIG. 2A is a perspective view from a distal end of a simplified sterile bag/drape in a folded non-deployed state, according to the first example embodiment of the disclosure.

FIG. 2B is a cross-sectional view A-A of the simplified sterile bag/drape in a folded non-deployed state without packaging, according to the first example embodiment of the disclosure.

FIG. 3A is a perspective view of an attaching end of the simplified sterile bag/drape in a folded non-deployed state, according to the first example embodiment of the disclosure.

FIG. 3B is a cross-sectional view A-A of the simplified sterile bag/drape in a folded non-deployed state without packaging, according to the first example embodiment of the disclosure.

FIG. 4A is an attaching end view of the simplified sterile bag/drape assembly depicted in a folded non-deployed state in which the simplified sterile bag/drape is hermetically sealed with packaging, according to the first example embodiment of the disclosure.

FIG. 4B is a cross-sectional view of a simplified sterile bag/drape assembly depicted in a folded non-deployed state in which the simplified sterile bag/drape is hermetically sealed with packaging, according to the first example embodiment of the disclosure.

FIG. 5A is a cross-sectional view of the simplified sterile bag/drape in a folded non-deployed state in which a portion of the packaging has been removed from the deployment port attaching end of the simplified sterile drape/packaging assembly before deployment, according to the first example embodiment of the disclosure.

FIG. 5B is a cross-sectional view of the simplified sterile bag/drape in a folded non-deployed state in which an attachment packaging portion has been removed from the deployment port attaching end of the simplified sterile bag/drape packaging assembly, according to the first example embodiment of the disclosure.

FIG. 6A is a cross-sectional view of the simplified sterile bag/drape in a folded non-deployed state in which the attachment packaging portion has been removed from the deployment port attaching end of the simplified sterile bag/drape packaging assembly and of which been attached to a mating portion on the deployment port, according to the first example embodiment of the disclosure.

FIG. 6B is a cross-sectional view of the simplified sterile bag/drape in a folded non-deployed state in which an inner liner packaging portion of the packaging has been removed, according to the first example embodiment of the disclosure.

FIG. 7A is a cross-sectional view of the simplified sterile bag/drape in a folded non-deployed state in which the inner liner packaging portion of the packaging has been removed with a medical instrument/device positioned internally in the deployment port and mated to an aperture/orifice formed in a distal tip of the simplified sterile bag/drape, according to the first example embodiment of the disclosure.

FIG. 7B is a cross-sectional view of the simplified sterile bag/drape in a folded non-deployed state in which the exterior portion of the packaging has been entirely removed with the medical instrument/device positioned internally in the deployment port and mated to the aperture formed in a distal tip of the simplified sterile bag/drape, according to the first example embodiment of the disclosure.

FIG. 8 is an example method for fully deploying the simplified sterile bag/drape assembly, according to the first example embodiment of the disclosure.

FIG. 9 is a cross-sectional view of the simplified sterile bag in a folded semi-deployed attached state in which the medical instrument is positioned internally within an instrument/device support disposed within the deployment port, according to another example embodiment of the disclosure.

### DETAILED DESCRIPTION OF THE DISCLOSURE

These and other aspects, features, and advantages of numerous example embodiments of the present disclosure will become apparent upon reading the following detailed description of the numerous example embodiments of the present disclosure, when taken in conjunction with the appended drawings, and provided claims.

Hereinafter, various modes to implement the disclosure will be described in detail with example embodiments with reference to drawing. Note that, dimensions, materials, shapes, and relative arrangement of components described in the example embodiments are to be appropriately modified in accordance with a configuration of an apparatus to which the disclosure is applied and various conditions, and the scope of the disclosure is not intended to be limited to the following example embodiments.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative example embodiments. It is intended that changes and modifications can be made to the described example embodiments without departing from the true scope and spirit of the subject disclosure as defined by the appended claims.

### First Example Embodiment of the Simplified Sterile Bag/Drape Assembly

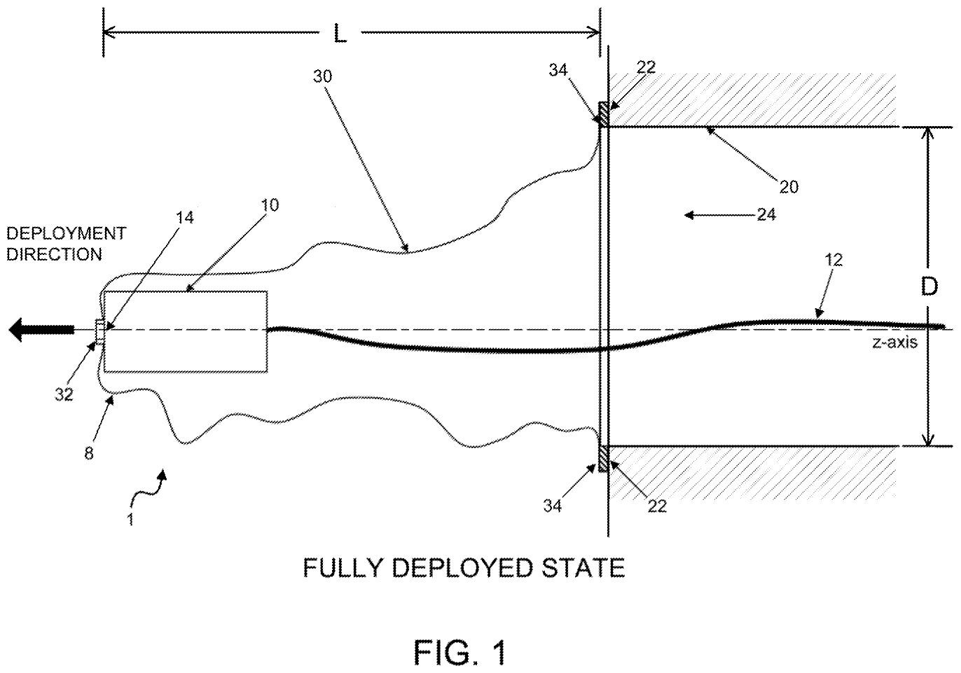

FIG. 1 is a cross-sectional view of a simplified sterile bag/drape 1 that has been fully deployed, according to a first example embodiment of the disclosure. The simplified sterile bag/drape 1 (hereinafter referred to “simplified sterile bag”) may be made of a foldable medical grade plastic film known in the art for medical and biological draping purposes. The example of the simplified sterile bag 1 in FIG. 1 is formed generally in a shape of an annular bag/drape 30 or (or tube, sheath or the like) having a diameter D to accept a medical instrument/device 10 (hereinafter referred to as “medical instrument” and having a length L sufficient to protect an entire sterile field when the medical device 10 is completely deployed. It is noted that the diameter D and length L illustrated in FIG. 1 are not necessarily to scale and instead provided merely for explanation purposes.

Moreover, in other embodiments, the bag/drape 30 may not have an annular shape, and instead, may have a rectangular, trapezoidal, oval, etc. cross-sectional shape formed to cover various medical system components having different shapes/sizes. As shown in FIG. 1, the bag/drape 30 may have a tapered shape or the like such that full coverage can be accomplished. Hence, it is noted that the shape of the bag/drape 30 can vary depending on the medical systems being utilized, and therefore, should not be limited to the shape illustrated in FIG. 1. And still further, it is noted that although an annular shape is described in the drawing/figures of the subject disclosure for description purposes, the subject disclosure should not be limited to only annular shaped bags/drapes 30.

The bag/drape 30 includes a deployment port attaching end 34 configured to be attached to a mating portion or attachment surface 22 (hereinafter referred to as “mating portion”) of a deployment port 24 that is part of a medical system. The mating portion 22 in FIG. 1 is on an exterior surface of the deployment port 24 that is part of a housing 20 of the medical system. That is to say the deployment port 24 can be formed in a housing 20 of the medical system as one example embodiment.

Furthermore, the deployment port 24 may be located elsewhere in a variety of places/positions/structures of the medical system, and is thus, should not be limited to the illustrated position in the disclosure. For example, the location of the mating portion 22 can also be on an internal surface of the housing 20 in another embodiment. Or in an alternative another alternative embodiment, the deployment port 24 may be located in an independent or standalone structure (not illustrated) designed for the sole purpose of supporting the simplified sterile bag 1. Or in other embodiments, the medical instrument 10 may rest on a top or side of a main console of the medical system. Accordingly, the medical instrument 10 does necessarily need to be deployed from the housing 20 of the medical system.

Still referring to FIG. 1, the bag/drape 30 also includes a distal deployment end 8 having a mating feature 32 such as an aperture/orifice and/or receptacle configured to receive a distal portion 14 of the medical instrument 10. Further details of the distal deployment end 8 and mating feature 32 of the simplified sterile bag 1 will be discussed later in the disclosure.

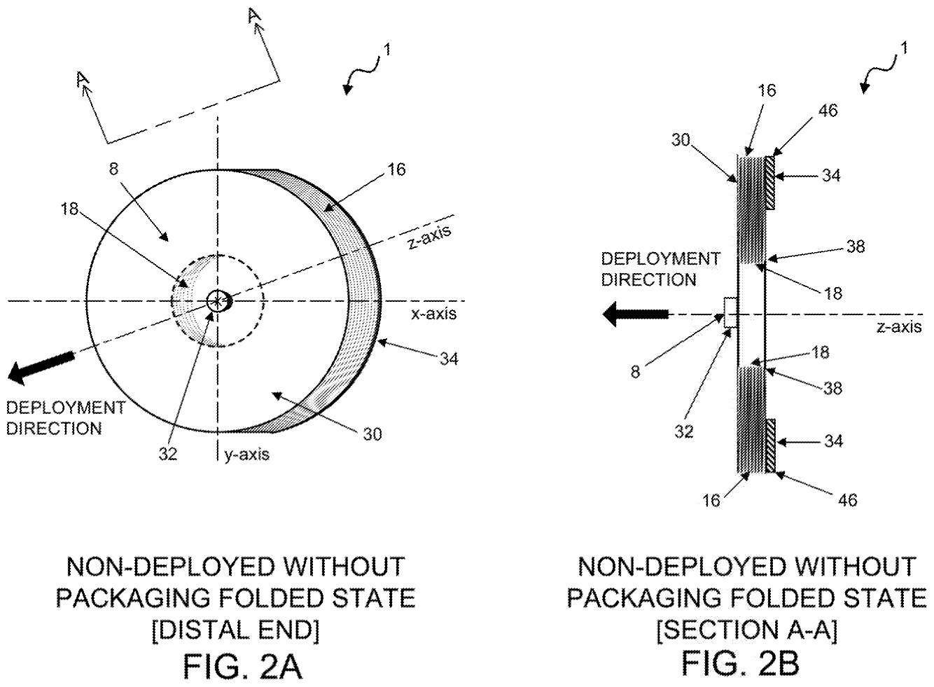

FIG. 2A is a perspective view of the distal deployment end 8 of the simplified sterile bag 1 in a folded non-deployed state without packaging, while FIG. 2B is a cross-sectional view A-A of the simplified sterile bag 1 in the folded non-deployed state without packaging, according to a first example embodiment of the disclosure. The bag/drape 30 drape is folded generally perpendicular to the direction of deployment of the medical instrument 10 thereby forming a cylindrically-shaped tube having an exterior radial wall 16 formed by exterior folds and an interior radial wall 18 formed by interior folds when the simplified sterile bag 1 is in a non-deployed state. It is further noted that other known methods for folding bags/drapes 30 may also be implemented in the numerous embodiments of the disclosure and therefore the disclosure should not be limited to the folding configuration illustrated in the drawings/figures.

Upon deployment, bag/drape 30 is generally concentrically unfolded about the center z axis when the medical instrument 10 is moved from the non-deployed position to the deployed position. However, it is noted that the deployment of the bag/drape 30 may also include being unfolded in varying directions about various axis (x-axis, y-axis or z-axis) and is not necessarily limited about the center z axis when the medical instrument 10 is moved from the non-deployed position to the deployed position. For example, as shown in FIG. 1, the medical instrument 10 is being deployed generally parallel or along the z-axis, but not directly about the z-axis.

Still referring to FIGS. 2A-B, it is noted that the attaching end 34 of the simplified sterile bag 1 includes an attaching means 46 for attaching the bag/drape 30 to the mating portion of the deployment port 24 which may implement various attaching means known in the art of bags/drapes 30 or any other extruded plastic covering (such as tubes, sheaths, etc). In one example embodiment, the attaching means 46 is adhesive pre-applied to the deployment port attaching end 34 for attaching the bag/drape 30 to the mating portion 22 of the deployment port 24. In another embodiment, the attaching means 46 is a thin magnetic flange or magnets affixed to the deployment port attaching end 34 of the bag/drape 30 for magnetically attaching to the mating portion 22 of the deployment port 24. And yet in other embodiments, the attaching means 46 may be any mechanical means known in the art suited for attaching and or coupling medical bags/drapes to support structures.

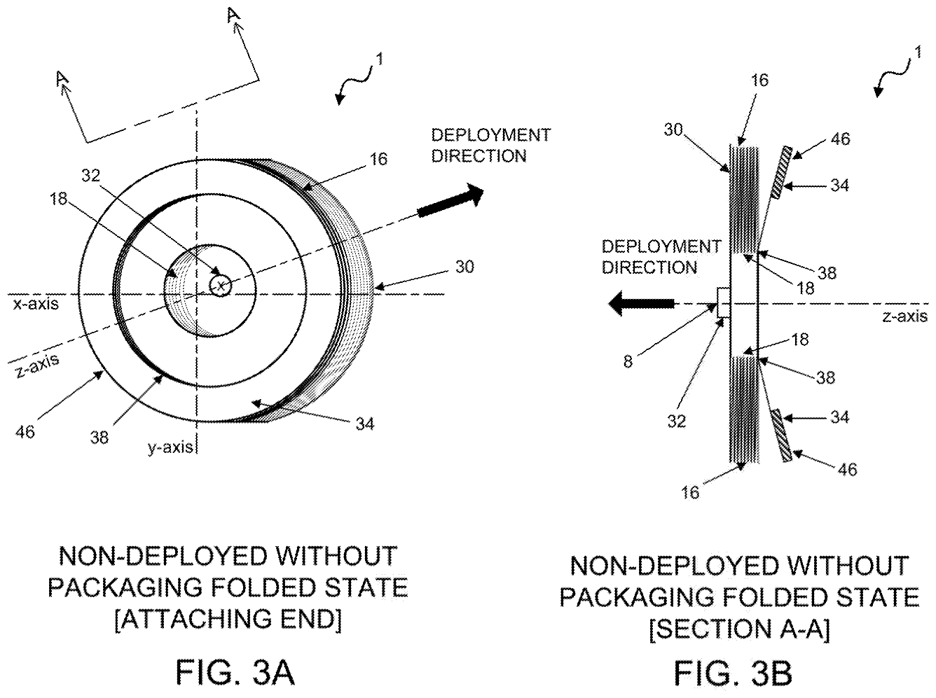

FIG. 3A is a perspective view of the deployment port attaching end 34 of the simplified sterile bag 1 in a folded non-deployed state without packaging, while FIG. 3B is a cross-sectional view A-A of the simplified sterile bag 1 in a folded non-deployed state without packaging, according to a first example embodiment of the disclosure. As shown in the example embodiment of FIG. 3B, the simplified sterile bag 1 in a folded non-deployed state without packaging forms an interior radial wall 18 formed by interior folds when the simplified sterile bag 1 is in a non-deployed state. In the embodiment shown in FIG. 3B, the attaching means 46 is applied exteriorly to the attaching end 34 of the bag/drape 30 towards the exterior radial wall 16 formed by exterior folds. In this embodiment, the last fold is therefore located inboard or along the interior radial wall 18. In another embodiment (not shown), the attaching means 46 is applied exteriorly to the attaching end 34 of the bag/drape 30 towards the interior radial wall 18 formed by interior folds. In this embodiment, the last fold is therefore located outboard or along the exterior radial wall 16. Thus, the location of the mating portion 22 can be on the internal surface of the housing 20 in this alternative embodiment.

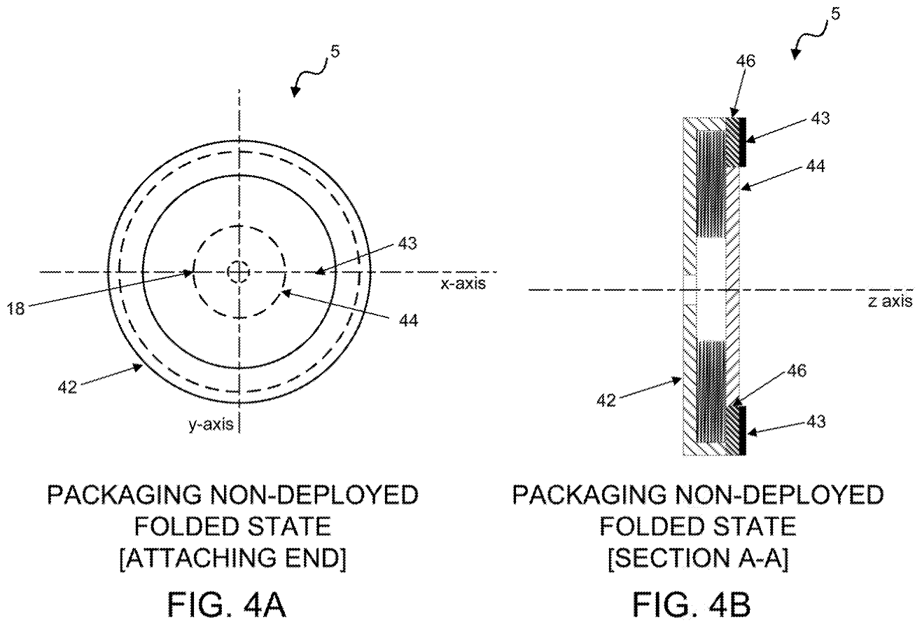

FIG. 4A is an attaching end view and FIG. 4B is a cross-sectional view of a simplified sterile bag and packaging assembly 5 in which the simplified sterile bag 1 is depicted in a folded non-deployed state and is hermetically sealed with packaging portions 42 (distal end exterior packaging), 43 (inner liner packaging) and 44 (attachment packaging), according to the first example embodiment of the disclosure. Such sterile hermetically sealed packaging portions 42, 43, 44 are applied over the simplified sterile bag to maintain sterility of the same before deployment. In the subject example embodiment depicted in FIGS. 4A-B, the packaging 42, 43, 44 is applied over the simplified sterile bag 1 such that plural portions of the packaging 42, 43, 44 may be removed in stages or steps before and during deployment of the simplified sterile bag.

Still referring to FIGS. 4A-B, the packaging portions 42, 43 and 44 will now be further described. As stated above, in the instant embodiment, packaging portions 42 (distal end exterior packaging), 43 (inner liner packaging) and 44 (attachment packaging), are configured such that each portion 42, 43 and 44 may be removed at different steps during deployment of the simplified sterile bag 1. This is accomplished by arranging the packaging such that the inner liner packaging 44 of the disclosed example embodiment is concentric to the attachment packaging 43, both of which are applied over the deployment port attaching end 34 of the simplified sterile bag 1. Additionally, the distal exterior packaging 42 is applied over the distal deployment end 8 of the simplified sterile bag 1. Moreover, in the illustrated embodiment shown in FIG. 4B, the distal exterior packaging 42 also covers the exterior radial wall 16 formed by the exterior folds of the bag/drape 30 in the non-deployed state. Thus, when the distal exterior packaging 42 is eventually removed last in a deployment sequence, the entire distal deployment end 8 of the simplified sterile bag 1 and the exterior or outboard folded edges 16 of the bag/drape 30 are exposed.

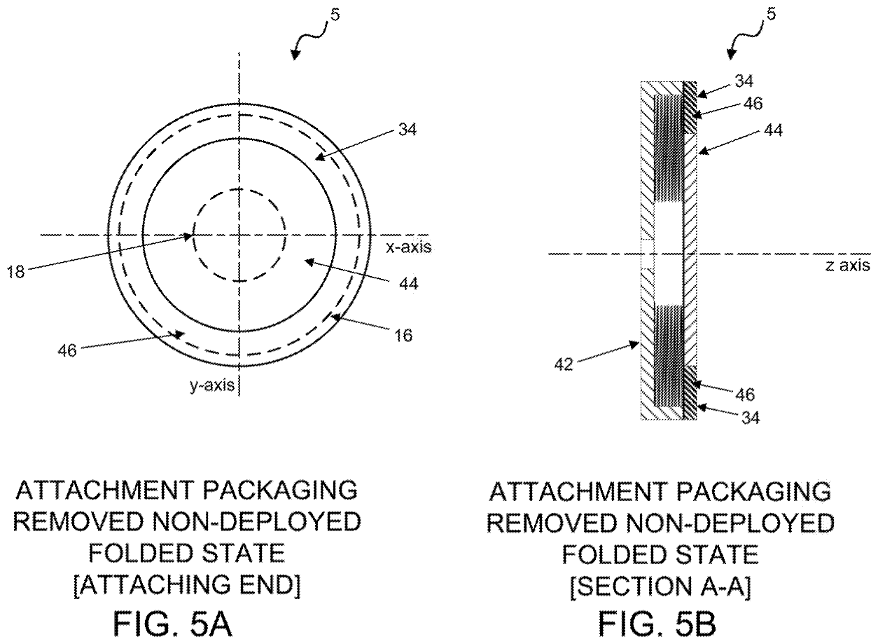

FIG. 5A is an attaching end view and FIG. 5B is a cross-sectional view of the simplified sterile bag 1 and packaging assembly 5 in a folded non-deployed state in which the attachment packaging 43 has been removed from the deployment port attaching end 34 of the simplified sterile bag 1 before deployment, according to the first example embodiment of the disclosure. The attachment packaging 43 is able to be first removed so that the deployment port attaching end 34 of the simplified sterile bag 1 can be attached to the mating portion 22 of the deployment port 24 while still maintaining/preserving sterile conditions for the simplified sterile bag 1 in an attached folded non-deployed state.

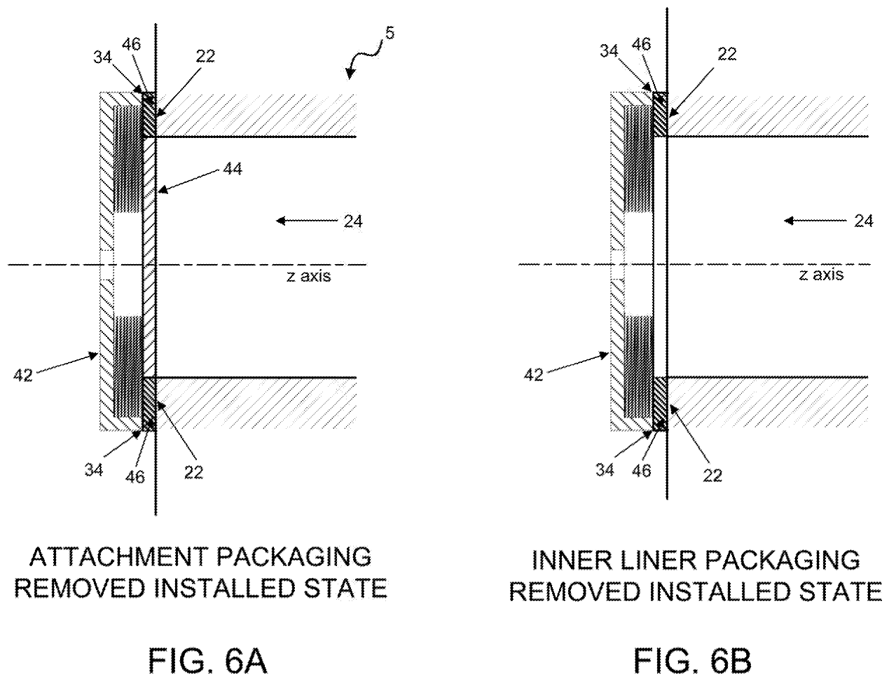

FIG. 6A is a cross-sectional view of the simplified sterile bag 1 and packaging assembly 5 in a folded non-deployed state in which the attachment packaging portion 43 has been removed from the deployment port attaching end 34 of the simplified sterile bag 1 and packaging assembly 5 and of which been attached to the mating portion 22 on the deployment port 24, according to the first example embodiment of the disclosure. Here, the attachment packaging 43 has been removed and the deployment port attaching end 34 of the simplified sterile bag 1 has been attached to the mating portion 22 of the deployment port 24 while still maintaining/preserving sterile conditions for the simplified sterile bag 1 packaging assembly 5 in an attached folded non-deployed state.

FIG. 6B is a cross-sectional view of the simplified sterile bag 1 and packaging assembly 5 in a folded non-deployed state in which the inner liner packaging portion 44 of the packaging has been removed from the deployment port attaching end 34 of the packaging assembly 5 before deployment, according to the first example embodiment of the disclosure. The inner liner packaging portion 44 is removed so that the distal portion 14 of the medical instrument 10 may be mated to the mating feature 32 such as an aperture/orifice and/or receptacle configured to receive the distal portion 14 of the medical instrument 10.

It is further noted that in another embodiment, the inner liner packaging portion 44 does not necessarily have to be removed. That is to say, the medical instrument 10 may be pierced through the inner liner packaging portion 44 so that the medical instrument 10 can be advanced through deployment port 24 with the inner liner packaging portion 44 left in place and not removed. In such embodiment, pre-scored lines may be formed in the inner liner packaging 44 to facilitate appropriate piercing through the inner liner packaging 44 portion.

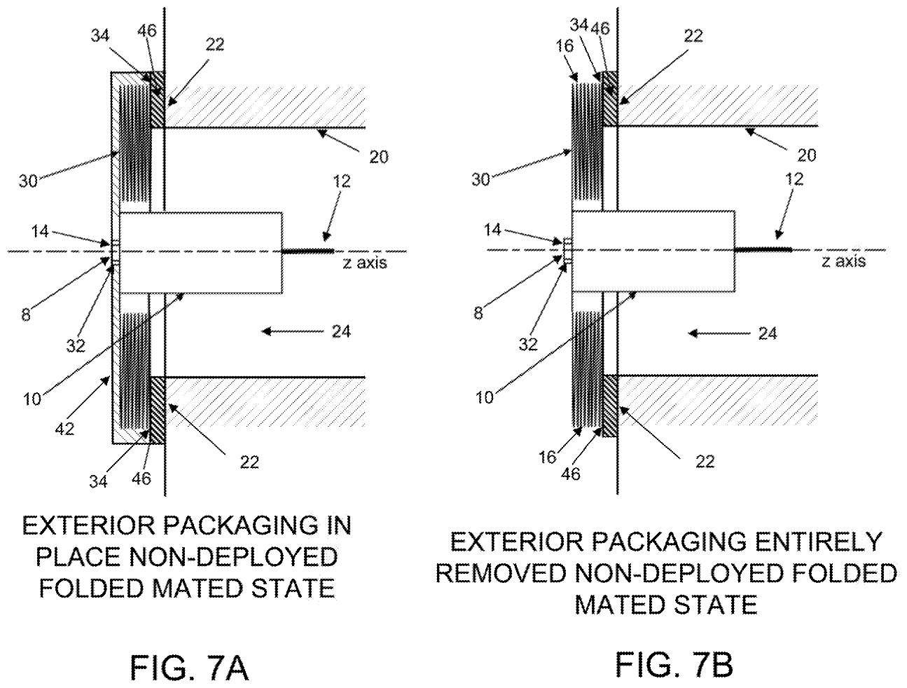

FIG. 7A is a cross-sectional view of the simplified sterile bag 1 and packaging assembly 5 in a folded non-deployed state in which the inner liner packaging portion 44 of the packaging has been removed with the medical instrument 10 positioned internally in the deployment port 24 and mated to the mating feature 32, such as an aperture/orifice/receptacle formed in a distal tip 8 of the simplified sterile bag 1, according to the first example embodiment of the disclosure. At this point in the deployment sequence, the distal tip 14 of the medical instrument 10 may be mated with the mating feature 32 having the aperture/orifice/receptacle affixed or formed integrally into to the distal deployment end 8 of the bag/drape 30. Since an inner surface of the bag/drape 30 does not have to maintain sterility and will get in contact with a non-sterile medical instrument 10, removal of the inner liner packaging portion 44 of the packaging may be performed by a non-sterile operator. Furthermore, since the exterior packaging portion 42 is still intact at his point in the deployment sequence, it will aid any medical instrument 10 mating manipulations by the non-sterile operator with the mating feature 32.

FIG. 7B is a cross-sectional view of the simplified sterile bag 1 in a folded non-deployed state in which the exterior packaging portion 42 of the packaging has been entirely removed from the simplified sterile bag 1 with the distal tip 14 of medical instrument 10 positioned internally in the deployment port 24 and mated to the mating feature 32 formed/integrated into the a distal tip 8 of the simplified sterile bag 1, according to the first example embodiment of the disclosure. At this point in the deployment sequence, an operator is now able to deploy the medical instrument 10 through the deployment port 24 such that the medical instrument 10 clears the deployment port such as illustrated in FIG. 1. Next, an example method for simplified deployment of the simplified sterile bag 1 by one person will herein be described below.

### Example Method for Simplified Deployment of the Simplified Sterile Bag/Drape Assembly by a Single Operator

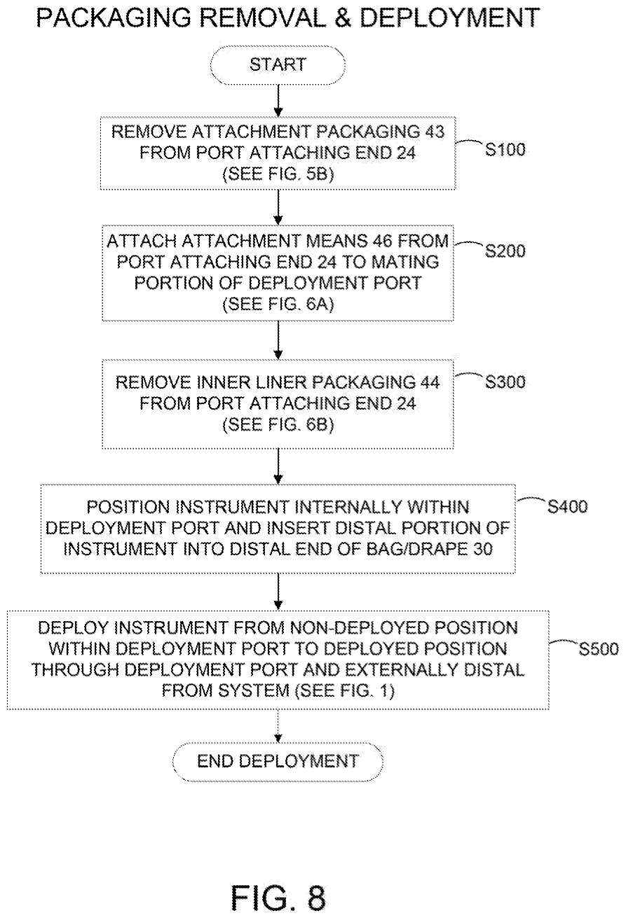

FIG. 8 is an example method for fully deploying the simplified sterile bag 1 and packaging assembly 5 by a single operator, according to the example first embodiment of the disclosure.

In Step 100, the simplified sterile bag 1 and packaging assembly 5 is in a folded non-deployed state completely hermetically sealed packaged state. First, the attachment packaging 43 is removed from the deployment port attaching end 34 of the simplified sterile bag 1 before deployment, according to the first example embodiment of the disclosure. The attachment packaging 43 is able to be first removed so that the deployment port attaching end 34 of the simplified sterile bag 1 can be attached to the mating portion 22 of the deployment port 24 while still maintaining/preserving sterile conditions for the simplified sterile bag 1 in an attached folded non-deployed state by a single operator. (See FIG. 5B).

Next in step 200, the simplified sterile bag 1 and packaging assembly 5 is configured in a folded non-deployed state in which the attachment packaging portion 43 has been removed by a single operator from the deployment port attaching end 34 of the simplified sterile bag 1 and packaging assembly 5 and of which been attached to the mating portion 22 on the deployment port 24 by a single operator, according to the first example embodiment of the disclosure. Here, the attachment packaging 43 is removed and the deployment port attaching end 34 of the simplified sterile bag 1 is attached to the mating portion 22 of the deployment port 24 while still maintaining/preserving sterile conditions for the simplified sterile bag 1 packaging assembly 5 in an attached folded non-deployed state by the single operator. (See FIG. 6A).

Next in step 300, the simplified sterile bag 1 and packaging assembly 5 is configured in a folded non-deployed state in which the inner liner packaging portion 44 of the packaging has been removed by a single operator from the deployment port attaching end 34 of the packaging assembly 5 before deployment, according to the first example embodiment of the disclosure. The inner liner packaging portion 44 is removed so that the distal portion 14 of the medical instrument 10 may be mated to the mating feature 32 such as an aperture/orifice and/or receptacle configured to receive the distal portion 14 of the medical instrument 10. (See FIG. 6B).

Next in step 400, the simplified sterile bag 1 and packaging assembly 5 in configured in a folded non-deployed state in which the inner liner packaging portion 44 of the packaging has been removed by a single operator with the medical device 10 positioned internally in the deployment port 24 and mated to the mating feature 32, such as an aperture/orifice/receptacle formed in a distal tip 8 of the simplified sterile bag 1, according to the first example embodiment of the disclosure. At this point in the deployment sequence, the distal tip 14 of the medical instrument 10 is mated by a single operator with the mating feature 32 having the aperture/orifice/receptacle affixed or formed integrally into to the distal deployment end 8 of the bag/drape 30. Since an inner surface of the bag/drape 30 does not have to maintain sterility and will get in contact with a non-sterile medical instrument 10, removal of the inner liner packaging portion 44 of the packaging may be performed by a single non-sterile operator. Furthermore, since the exterior packaging portion 42 is still intact at his point in the deployment sequence, it will aid any medical instrument 10 mating manipulations by the single non-sterile operator with the mating feature 32. (See FIG. 7A).

Next in step 500, the simplified sterile bag 1 is configured in a folded non-deployed state in which the exterior packaging portion 42 of the packaging has been entirely removed by a non-sterile operator from the simplified sterile bag 1 and with the distal tip 14 of medical instrument 10 being positioned internally in the deployment port 24 and mated by a non-sterile operator to the mating feature 32 formed/integrated into the a distal tip 8 of the simplified sterile bag 1, according to the first example embodiment of the disclosure.

Next in step 600, the medical instrument 10 is ready to be deployed by a sterile operator through the deployment port 24 such that the medical instrument 10 clears the deployment port 24, according to the first example embodiment of the disclosure.

In an alternative method embodiment, if all the above steps are performed by a single operator, the single operator will required sterile cleaning/scrubbing before proceeding with step 600.

As can be understood from the above description, the present disclosure allows for simplified sterile bag/drape 1 deployment coincident and combined with the medical instrument 10 deployment saving significant portion of operator (especially sterile operator) time. The present disclosure allows for simplified sterile bag/drape 1 to be easily deployed by one person and/or by a multiple person team, due to desynchronized actions of sterile and non-sterile operators.

Accordingly, a simplified sterile bag/drape with packaging and method for simplified deployment of the same is realized from the present disclosure wherein the simplified sterile bag 1 may be easily deployed from a medical system by only one person or by a multiple person team, due to desynchronized actions of sterile and non-sterile operators, thereby reducing operating expenses.

### Other Example Aspects, Features and Embodiments

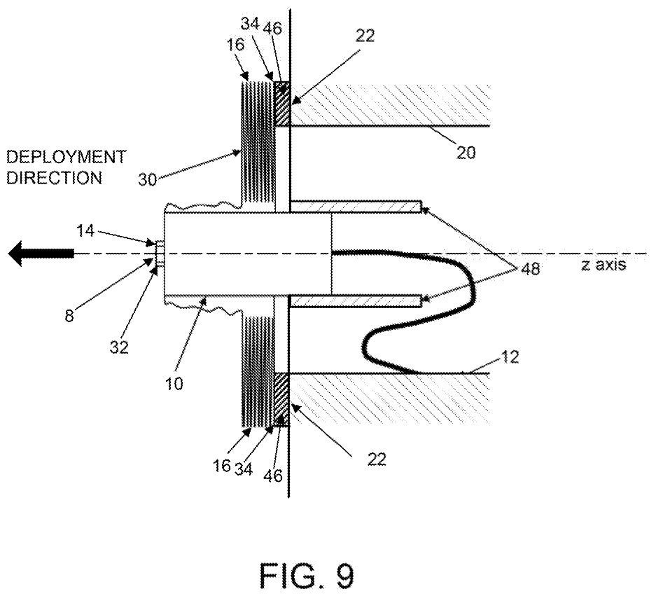

FIG. 9 is a cross-sectional view of the simplified sterile bag 1 in a folded semi-deployed attached state in which the packaging 42, 43, 44 has been removed. Here, the medical instrument 10 is positioned internally within an instrument/device support 48 disposed within (or partially within) the deployment port 24 and mated to the mating feature 32 integrated in a distal tip 8 of the simplified sterile bag 1, according to another example embodiment of the disclosure. With this arrangement, the medical instrument 10 may rest/slide in the instrument/device support 48 so as to allow for some initial movement (push from the deployment port 24) of the medical instrument 10 by a non-sterile operator in the deployment direction to facilitate better grip for further pulling of the medical instrument 10 by a sterile operator.

Yet in another alternative embodiment, such an instrument/device support 48 may include a linear slide or an articulated arm (not shown) deployable in conjunction with the medical instrument/device 10 inside the simplified sterile bag 1 to provide for additional device support and/or articulation during use.

Moreover, in another alternative embodiment, an additional feature (such as a handle or gripping feature) may be added to an outer sterile surface of the mating feature 32 or elsewhere on the bag assembly 1 to facilitate medical instrument 10 deployment of the simplified sterile drape 1 by a sterile operator during deployment and subsequent handling and manipulating of the medical instrument 10.

While yet in another alternative embodiment, where a sterile probe, such as a cardiovascular imaging catheter, is to be attached to the medical instrument 10, a corresponding opening/receptacle/adaptor may be provided in the distal end 8 of the simplified sterile bag 1 to allow a probe attachment portion to mate with the medical instrument 10 so that each are securely engaged/connected together. The probe attachment portion may be further adapted for convenient grip (as a handle) during deployment and subsequent handling and manipulating of the medical instrument 10.

Or in an alternative method embodiment, the inner liner packaging portion 44 is left in place by the single operator. Instead, the medical instrument 10 is pierced through the inner liner packaging portion 44 so that the medical instrument 10 can be advanced through deployment port 24 with the inner liner packaging portion 44 left in place and not removed. In such embodiment, pre-scored lines may be formed in the inner liner packaging 44 to facilitate appropriate piercing through the inner liner packaging 44 portion.

Or in an alternative method embodiment, the inner liner packaging portion 44 is omitted all together the and the annular bag/drape 30 itself functions as a sterile barrier.

In the embodiment shown in FIG. 3B, the attaching means 46 is applied exteriorly to the attaching end 34 of the bag/drape 30 towards the exterior radial wall 16 formed by exterior folds. In this embodiment, the last fold is therefore located inboard or along the interior radial wall 18. While yet in another embodiment, the attaching means 46 is applied exteriorly to the attaching end 34 of the bag/drape 30 towards the interior radial wall 18 formed by the interior folds. With respect to the latter alternative embodiment, the last fold 38 is formed along the exterior radial wall 18. Accordingly, such configuration allows for the deployment port attaching end 34 to be attached on an inside surface 20 of the deployment port 24 since the attaching means 46 is located on an exterior side of the bag/drape 30 and further since the mating portion 22 of the deployment port 24 is located internally within the deployment port.

Additionally, in another alternative embodiment, the simplified sterile bag 1 and packaging assembly 5, may be inserted and retained in place by being installed, after removal of attachment packaging portion 43, into a corresponding sized recess or slot formed in the structure defining the deployment port 24. In this configuration, since no portion of the simplified sterile bag 1 and packaging assembly 5 is yet exposed before removal of attachment packaging portion 43, this step it may be performed by a non-sterile personal.

The foregoing merely illustrates the principles of the disclosure. Various modifications and alterations to the described exemplary embodiments will be apparent to those skilled in the art in view of the teachings herein.

It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

While the disclosure has been described with reference to example embodiments, it is to be understood that the present disclosure is not limited to the disclosed example embodiments. The scope of the following claims is to be accorded the broadest interpretation so as to encompass all such modifications and equivalent structures and functions.

What is claimed:

1. A contamination prevention bag assembly for covering an instrument deployable from a system having a deployment port for deploying the instrument, the instrument being deployable from a non-deployed position to a deployed position through the deployment port externally distal from the deployment port, the bag assembly comprising:
a bag comprising:
an inner and an outer bag surface;
a deployment port attaching end configured to be attached to a mating portion of the deployment port; and
a distal deployment end having a distal mating feature configured to receive a distal portion of the instrument,
wherein the bag is folded in a non-deployed state,
wherein the bag is unfolded when the instrument is moved from the non-deployed position to the deployed position.

2. The contamination prevention bag assembly according to claim 1,
further comprising an attachment means disposed on the deployment port attaching end of the bag so that the deployment port attaching end is configured to be removably attached to the mating portion of the deployment port.

3. The contamination prevention bag assembly according to claim 2,
wherein the attachment means includes adhesive pre-applied to the deployment port attaching end for attaching the bag to the mating portion of the deployment port.

4. The contamination prevention bag assembly according to claim 2,
wherein the attachment means includes a magnetic flange affixed to the deployment port attaching end that magnetically attaches to the mating portion of the deployment port.

5. The contamination prevention bag assembly according to claim 2,
wherein the attachment means includes a mechanical means for removably attaching the deployment port attaching end of the bag to the mating portion of the deployment port.

6. The contamination prevention bag assembly according to claim 2,
further comprising contamination prevention packaging hermetically sealing at least the outer bag surface in a non-deployed state,
wherein the bag is packaged and stored in a folded state.

7. The contamination prevention bag assembly according to claim 6,
wherein the contamination prevention packaging includes at least one removable attachment portion covering the deployment port attaching end of the non-deployed drape assembly, and
wherein the at least removable portion is configured to be removed before attaching the attachment means to the mating portion of the deployment port.

8. The contamination prevention bag assembly according to claim 6,
wherein the contamination prevention packaging includes at least one removable inner liner covering the deployment port attaching end of the non-deployed drape assembly, and
wherein the at least one removable inner liner portion is configured to be removed before attaching the distal portion of the instrument to the distal mating feature configured to receive the distal portion of the instrument.

9. The contamination prevention bag assembly according to claim 6,
wherein the contamination prevention packaging includes at least one removable portion covering at least a distal side and a length side of the bag assembly defined by folded edges formed by the folded bag in the non-deployed state, which when removed, uncovers the distal deployment end of the bag assembly and the folded edges of the bag.

10. A method for utilizing a contamination prevention bag assembly for covering an instrument deployable from a system having a deployment port,
the bag assembly comprising:
a bag folded in a non-deployed state, the bag including inner an inner and outer surface, a deployment port attaching end, a distal deployment end having a mating feature configured to receive a distal portion of the instrument, and an attachment means disposed on the deployment port attaching end of the bag; and
contamination prevention packaging hermetically sealing at least the outer bag surface in a non-deployed state,
the method comprising:
attaching the attachment means of the bag assembly to a mating portion of the deployment port;
inserting the distal portion of the instrument through the deployment port attaching end of the bag assembly and engaging the distal portion of the instrument into the distal mating feature configured to receive a distal portion of the instrument;
removing the contamination prevention packaging hermetically sealing at least the outer bag surface; and
deploying the instrument from a non-deployed position to a deployed position through the deployment port and externally distal from the deployment port,
wherein the bag is unfolded when the instrument is moved from the non-deployed position to the deployed position externally distal from the deployment port.

11. The method for utilizing a contamination prevention bag assembly according to claim 10, the contamination prevention packaging further comprising:
at least one removable portion covering at least a distal side and a length side of the bag assembly defined by folded edges formed by the folded bag in the non-deployed state,
the method further comprising:
removing the packaging from the at least one removable portion covering the distal deployment end side and the length side of the bag assembly so that the distal deployment end and the folded edges of the bag assembly are uncovered.

12. The method for utilizing a contamination prevention bag assembly according to claim 10, the contamination prevention packaging further comprising:
at least one removable attachment portion covering the deployment port attaching end of the non-deployed drape assembly,
the method further comprising:
removing the at least removable portion before attaching the attachment means to the mating portion of the deployment port.

13. The method for utilizing a contamination prevention bag assembly according to claim 10, the contamination prevention packaging further comprising:
at least one removable inner liner covering the deployment port attaching end of the non-deployed drape assembly,
the method further comprising:
removing the at least one removable inner liner portion before attaching the distal portion of the instrument to the distal mating feature configured to receive the distal portion of the instrument.

14. The method for utilizing a contamination prevention bag assembly according to claim 10,
wherein the attachment means includes adhesive pre-applied to the deployment port attaching end of the bag assembly for attaching the bag assembly to the mating portion of the deployment port.

15. The method for utilizing a contamination prevention bag assembly according to claim 10,
wherein the attachment means includes a magnetic flange affixed to the deployment port attaching end that magnetically attaches to the mating portion of the deployment port.

16. The method for utilizing a contamination prevention bag assembly according to claim 10,
wherein the attachment means includes a mechanical means for removably attaching the deployment port attaching end of the sheath to the mating portion of the deployment port.

17. A contamination prevention bag assembly for covering an instrument deployable from a system having a deployment port for deploying the instrument, the instrument being deployable from a non-deployed position to a deployed position through the deployment port externally distal from the deployment port, the bag assembly consisting:
a bag consisting:
an inner and an outer bag surface;
a deployment port attaching end with an attachment means disposed on the deployment port attaching end so that the deployment port attaching end is configured to be removably attached to the mating portion of the deployment port; and
a distal deployment end having a distal mating feature configured to receive a distal portion of the instrument; and
contamination prevention packaging hermetically sealing at least the outer bag surface in a non-deployed state,
wherein the bag is folded in a non-deployed state,
wherein the bag is unfolded when the instrument is moved from the non-deployed position to the deployed position.

18. A method for utilizing a contamination prevention bag assembly for covering an instrument deployable from a system having a deployment port,
the bag assembly consisting:
a bag consisting:
an inner and an outer bag surface;
a deployment port attaching end with an attachment means disposed on the deployment port attaching end so that the deployment port attaching end is configured to be removably attached to the mating portion of the deployment port; and
a distal deployment end having a distal mating feature configured to receive a distal portion of the instrument; and
contamination prevention packaging hermetically sealing at least the outer bag surface in a non-deployed state,
wherein the bag is folded in a non-deployed state,
wherein the bag is unfolded when the instrument is moved from the non-deployed position to the deployed position,
the method consisting:
attaching the attachment means of the bag assembly to a mating portion of the deployment port;
inserting the distal portion of the instrument through the deployment port attaching end of the bag assembly and engaging the distal portion of the instrument into the distal mating feature configured to receive a distal portion of the instrument;
removing the contamination prevention packaging hermetically sealing at least the outer bag surface; and
deploying the instrument from a non-deployed position to a deployed position through the deployment port and externally distal from the deployment port,
wherein the bag is unfolded when the instrument is moved from the non-deployed position to the deployed position externally distal from the deployment port.
